(19) United States
(12) Patent Application Publication
(10) Pub. No.: US 2023/0405560 A1
(43) Pub. Date: Dec. 21, 2023

(54) Chrome-Free Copper-Calcium Silicate Catalysis for Fatty Ester Hydrogenolysis/Hydrogenation

(71) Applicant: BASF CORPORATION, Florham Park, NJ (US)

(72) Inventors: Jian-Ping Chen, Beachwood, OH (US); Arunabha Kundu, Beachwood, OH (US)

(21) Appl. No.: 18/035,205

(22) PCT Filed: Nov. 3, 2021
(86) PCT No.: PCT/US2021/072206

§ 371 Date: May 3, 2023

Related U.S. Application Data
(60) Provisional application No. 63/109,591, filed on Nov. 4, 2020.

(51) Int. Cl. B01J 23/72; B01J 21/08; B01J 23/04; B01J 35/02; B01J 35/00; B01J 35/10; B01J 6/00; B01J 37/03; B01J 37/04; B01J 37/08

(52) U.S. Cl. CPC B01J 23/72; B01J 21/08; B01J 23/04; B01J 35/023; B01J 37/08; B01J 35/1014; B01J 6/001; B01J 37/031; B01J 37/04; B01J 35/006

(57) ABSTRACT

A hydrogenolysis/hydrogenation catalyst includes copper oxide, calcium oxide, silicon dioxide, and sodium oxide, wherein the hydrogenolysis/hydrogenation catalyst is a powder, tablet, or extrudate, the hydrogenolysis/hydrogenation catalyst is substantially free of chromium, the hydrogenolysis/hydrogenation catalyst exhibits a crystallite phase of CuO and an additional crystallite phase selected from the group consisting of cubic SiO₂, rhombohedral calcium carbonate CaCO₃, anorthic calcium silicate CaCO₃, calcium silicate hydroxide hydrate (Ca₁₄Si₂₄O₅₈(OH)₈·5H₂O), calcium silicate hydrate 4CaO·5 SiO₂·5H₂O, alumina, and combinations of two or more thereof.

### CROSS REFERENCE TO RELATED APPLICATION(S)

The present application claims priority to U.S. Provisional Application No. 63/109,591 filed on Nov. 4, 2020, the entire contents of which is incorporated by reference in its entirety.

### TECHNOLOGY

The present technology relates generally to the field of catalysts for hydrogenolysis/hydrogenation. More specifically, it is related to copper-calcium silicate-based catalysts in powder form slurry phase for fatty acid ester hydrogenolysis/hydrogenation.

### BACKGROUND

Commercial slurry processes for producing fatty alcohols typically employ copper-chromium (CuCr) catalysts. As environmental regulation becomes stricter on chromium containing chemicals or catalysts, it is crucial to develop catalysts that do not contain chromium but instead utilize other materials as both chemical and mechanical stability promoters and support.

### SUMMARY

In one aspect, a hydrogenolysis/hydrogenation catalyst includes copper oxide, calcium oxide, silicon dioxide, and sodium oxide, wherein the hydrogenolysis/hydrogenation catalyst is a powder and the hydrogenolysis/hydrogenation catalyst is substantially free of chromium. In various embodiments, the calcined hydrogenolysis/hydrogenation catalyst includes CuO from about 35 wt % to about 85 wt %, CaO from about 8 wt % to about 20 wt %, SiO₂ from about 10 wt % to about 30 wt %, and Na2O from about 0.1 wt % to about 5 wt %.

In another aspect, a method of preparing a calcined hydrogenolysis/hydrogenation catalyst is provided, the method including mixing a copper-containing material and silicate-containing material in a solution; adding a caustic material to form an aqueous slurry comprising a precipitate; collecting the precipitate; drying the precipitate to form a dried precipitate; and calcining the dried precipitate to form the calcined hydrogenolysis/hydrogenation catalyst; wherein the calcined hydrogenolysis/hydrogenation catalyst is a powder; and the calcined hydrogenolysis/hydrogenation catalyst is substantially free of chromium. A calcined hydrogenolysis/hydrogenation catalyst prepared according to the method is also provided.

In a further aspect, a method of hydrogenating a carbonyl-containing organic compound is provided, the method including contacting the carbonyl-containing organic compound with the hydrogenolysis/hydrogenation catalyst that catalyst includes copper oxide, calcium oxide, silicon dioxide, and sodium oxide, wherein the hydrogenolysis/hydrogenation catalyst is a powder, and the hydrogenolysis/hydrogenation catalyst is substantially free of chromium.

### BRIEF DESCRIPTION OF THE DRAWINGS

FIG. 1 is a graph of particle size formation as a function of precipitation time based upon “Volume In,” according to Example 1. Each plot shows the Cumulative Volume % (vol %) distribution of particles detected via laser diffraction within a particle size range.

FIG. 2 is a graph of particle size formation as a function of precipitation time based upon “percent passing,” according to Example 1. Each plot shows a normal distribution curve in which a majority of the sample volume is occupied by the mid-range sized particles (i.e., Volume %).

FIG. 3 illustrated x-ray diffraction (XRD) patterns of current inventive catalysts, according to Examples 2, 5, 7, and 8, and which have calcination temperature of 500, 650, 750, and 800° C., respectively.

FIG. 4 is a schematic diagram of an autoclave reactor used for catalyst performance testing, according to various embodiments.

FIG. 5 is a graphical comparison of methyl ester conversion (percentage) as a function of time for a standard CuCr catalyst, an CuAl catalyst, and the described CuO on a calcium silicate catalyst (Example 1), according to various embodiments.

FIG. 6 is a graphical comparison of fatty alcohol selectivity (percentage) as a function of time for a standard CuCr catalyst, a CuAl catalyst, and the described CuO on calcium silicate catalyst (Example 1), according to various embodiments.

FIG. 7 is a graphical comparison of fatty alcohol yield (percentage) as a function of time for a standard CuCr catalyst, a CuAl catalyst, and the described CuO on calcium silicate catalyst (Example 1), according to various embodiments.

FIG. 8A is a graphical comparison of methyl ester conversion (percentage) as a function of time, according to the examples.

FIG. 8B is a graphical comparison of methyl ester conversion (percentage) as a function of time, according to the examples, with a focus on the high conversion section.

FIG. 9A is a graphical comparison of fatty alcohol selectivity (percentage) as a function of time, according to the examples.

FIG. 9B is a graphical comparison of fatty alcohol selectivity (percentage) as a function of time, according to the examples, with a focus on the high selectivity section.

FIG. 10A is a graphical comparison of fatty alcohol yield (percentage) as a function of time, according to the examples.

FIG. 10B is a graphical comparison of fatty alcohol yield (percentage) as a function of time, according to the examples, with a focus on the high yield section.

FIG. 11 is a graphical comparison of methyl ester conversion (percentage) as a function of time, according to the examples with different Na content.

FIG. 12 is a graphical comparison of fatty alcohol selectivity (percentage) as a function of time, according to the examples with different Na content.

FIG. 13 is a graphical comparison of fatty alcohol yield (percentage) as a function of time, according to the examples with different Na content.

FIG. 14 is a graph of the catalyst performance in a wax ester slurry process (feed was injected at 0^{th} 1^{st} 2^{nd} and 3^{rd} hour) for a standard CuCr catalyst, a CuAl catalyst, and the described CuO on calcium silicate catalyst (Example 3), according to the examples. The higher the SAP value, the lower the conversion.

FIG. 15 is a graph of cumulative wax ester conversion for a standard CuCr catalyst, a CuAl catalyst, and the described CuO on calcium silicate catalyst (Example 3), according to various embodiments.

FIG. 16 is a graph illustrating the results of a filterability test on samples using a CuCr reference catalyst, a CuAl reference catalyst and the described CuO on calcium silicate catalyst (Example 1) in a methyl ester slurry process.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “hydrogenolysis/hydrogenation” refers to a catalyst, which in a particular application may catalyze either or both of a hydrogenolysis reaction and/or a hydrogenation reaction.

As used herein, the term “calcine,” “calcined,” or “calcining” refers to heating a catalyst precursor precipitate, in some embodiments as a dried filter cake, in an oven under an air or a controlled oxygen atmosphere.

As used herein, “substantially free” is intended to indicate that, to the extent possible, the material being described is excluded from the formulation. However, trace amounts may be carried through due to contamination in starting reagents. For example, where the term used is “substantially free of chromium,” it is intended that all chromium is to be ideally excluded, however, trace amounts of chromium may be carried due to contamination by chromium of the other starting reagents such as the copper, manganese, and aluminum source materials. For example, in some embodiments, “substantially free of chromium” may include less than 1000 ppm chromium, such as less than 750 ppm chromium, less than 500 ppm chromium, or less than 100 ppm chromium. Where appropriate, substantially free of chromium means that the catalyst contains no detectable chromium (0.0 wt % chromium). Similarly, the term is also applied to manganese, in some embodiments.

It has now been discovered that chromium-free, copper-calcium silicate powder catalysts may be prepared for slurry phase hydrogenolysis applications, and, in many respects, these catalysts have been determined to out-perform the state of the art copper-chrome catalysts currently used in commercial settings. For example, the powder catalysts described herein possess superior catalytic performance in terms of activity and selectivity, they exhibit reduced metal leaching, and have comparable stability under reaction conditions when compared to the state of the art Cu-chrome catalyst.

In one aspect, a hydrogenolysis/hydrogenation catalyst is provided, the catalyst comprising copper(II)oxide (“CuO”) on a calcium silicate powder (i.e. the catalyst are copper calcium silicate materials). The catalysts are active for hydrogenation or hydrogenolysis of compounds having a carbonyl moiety, particularly for methyl ester and wax ester hydrogenolysis reactions. The hydrogenolysis/hydrogenation catalyst include about 35 wt % to about 85 wt % CuO, about 8 wt % to about 20 wt % CaO, about 10 wt % to about 30 wt % SiO₂, about 0.1 wt % to about 5 wt % Na₂O, about 0 wt % to about 1.5 wt % Al₂O₃, and about 0 wt % to about 1 wt % K₂O. This includes in some embodiments, where the hydrogenolysis/hydrogenation catalyst includes about 35 wt % to about 70 wt % CuO, about 8 wt % to about 16 wt % CaO, about 15 wt % to about 22 wt % SiO₂, about 0.1 wt % to about 0.75 wt % Na₂O, 0 to about 1.5 wt % Al₂O₃, and 0 to about 0.75 wt % K₂O. The hydrogenolysis/hydrogenation catalysts are substantially free of chromium. While in some embodiments, the calcium silicate catalysts may include manganese, in other embodiments, the catalyst is substantially free of manganese.

The catalyst exhibits a crystalline phase of CuO, and one or more of the crystalline phases from the list CaCO₃, SiO₂, CaSiO₃, Ca₁₄Si₂₄O₅₈(OH)₈·2H₂O (Truscottite, or calcium silicate hydroxy hydrate), 4CaO·5SiO₂·5H₂O (Torbermorite or calcium silicate hydrate), and Al₂O₃. In some embodiments, the calcined hydrogenolysis/hydrogenation catalyst exhibits CuO and an additional crystallite phase selected from the group consisting of cubic SiO₂, rhombohedral calcium carbonate CaCO₃, anorthic calcium silicate CaSiO₃, calcium silicate hydroxide hydrate (Ca₁₄Si₂₄O₅₈(OH)₈·2H₂O), calcium silicate hydrate 4CaO·5SiO₂·5H₂O, alumina, and combinations of two or more thereof The Brunauer-Emmett-Teller Surface Area (BET SA) is from about 20 m 2 /g to about 100 m 2 /g. In some embodiments, the BET SA is from about 5 m²/g to about 85 m²/g.

The copper calcium silicate catalysts may be in the form of a powder. An average particle size of the powder may be described according to the following particle size distribution (“PSD”): D₁₀ about 1 μm to about 10 μm, D₅₀ from about 10 μm to about 25 μm microns, and D₉₀ from about 30 μm to about 45 μm. This may include a PSD of: D₁₀ about 1 μm to about 1.5 μm, D₅₀ from about 16 μm to about 20 μm microns, and D₉₀ from about 30 μm to about 35 μm. The loose packed bulk density of the copper-calcium silicate powder catalysts is from about 0.25 g/ml to about 0.6 g/ml, with a CuO crystallite size of about 50 Å to about 250 Å. In some embodiments, the CuO crystallite size is from about 50 Å to less than 240 Å. Methods for the production of the copper-calcium silicate catalysts and their use in hydrogenolysis/hydrogenation reactions is also described.

It is also noteworthy that filtration properties are important in fatty alcohol production when using slurry phase processes, as the catalysts must be separated from the reactor slurry for reuse, and to allow for pure fatty alcohol products. The copper-calcium silicate powder catalysts described herein, exhibit good filtration properties that are similar to those of the Cu-chrome state of the art materials. A catalyst with good filtration/separation properties will enable the fatty alcohol producing plant a high production throughput.

In another aspect, a method of preparing a calcined hydrogenolysis/hydrogenation catalyst is provided. The method includes mixing a copper-containing material and silicate-containing material in a solution, adding a caustic material to form an aqueous slurry comprising a precipitate, collecting the precipitate, drying the precipitate to form a dried powder; and calcining the dried powder to form the calcined hydrogenolysis/hydrogenation catalyst. In various embodiments, the calcined hydrogenolysis/hydrogenation catalyst may be a powder. In any of the above embodiments, the calcined hydrogenolysis/hydrogenation catalyst may be substantially free of chromium.

According to the methods, the aqueous slurry has a pH of about 6.0 to about 9.0. This includes a pH from about 7 to 7.5.

In the methods, the collection may be via filtration of the aqueous slurry to remove and collect the precipitate as a filter cake. The precipitate may also be washed with water to remove some of the sodium from filter cake. The washings may be conducted with large volumes of water and may be conducted repeated times (two, three, four, or more washings).

In the method of preparation, the drying of the precipitate may be done in an oven in a heated atmosphere. The heating may be from about 40° C. to about 200° C., from about 75° C. to about 150° C., or from about 100° C. to about 125° C. The drying may be done for a time period to ensure a dried powder. According to various embodiments, the time period may be from 1 hour to 24 hours, or more. This includes about 5 hours to about 15 hours, or about 8 hours to about 12 hours. In some embodiments, the drying is overnight.

In any of the above embodiments, the calcining may be conducted at a temperature from about 400° C. to about 800° C. This may include from about 500° C. to about 800° C., from about 500° C. to about 750° C., or from about 600° C. to about 750° C. The calcining may be done for a time period to complete calcination of the dried powder. According to various embodiments, the time period may be for about 10 minutes to about 10 hours. This includes from about 0.5 hour to about 3 hours.

In any of the above embodiments of the method, the copper-containing material may be a copper salt comprising copper nitrate, copper sulfate, copper chloride, copper bromide, copper acetate, or a combination of any two or more thereof. Similarly, the silicate-containing material may be a silicate salt comprising calcium silicate.

In any of the above embodiments of the method, the caustic material may be any caustic material. Illustrative caustics include, but are not limited to, Na₂CO₃, NaOH, K₂CO₃, KOH, or a combination of any two or more thereof.

The copper calcium silicate catalysts may be in the form of a powder. An average particle size of the powder may be described according to the following particle size distribution (“PSD”): D₁₀ from about 1 μm to about 10 μm, D₅₀ from about 10 μm to about μm microns, and D₉₀ from about 30 μm to about 45 μm. Where the catalysts are in the form of a powder, the average particle size may be described according to the following particle size distribution (“PSD”): D₁₀ about 1 μm to about 10 μm, D₅₀ from about 10 μm to about 25 μm microns, and D₉₀ from about 30 μm to about 45 μm. This may include a PSD of: D₁₀ about 1 μm to about 1.5 μm, D₅₀ from about 16 μm to about 20 μm microns, and D₉₀ from about 30 μm to about 35 μm. In some embodiments, D₁₀ may be from about 1 μm to about 2 μm, or from about 4 μm to about 9 μm. In some embodiments, D₅₀ may be from about 10 μm to about 25 μm, or from about 16 μm to about 20 μm. In some embodiments, D₉₀ may be from about 30 μm to about 45 μm, or from about 35 μm to about 40 μm. The loose packed bulk density of the copper-calcium silicate powder catalysts is from about 0.25 g/ml to about 0.6 g/ml, with a CuO crystallite size of about 50 Å to about 250 Å. In some embodiments, the CuO crystallite size is from about 50 Å to less than 240 Å. Methods for the production of the copper-calcium silicate catalysts and their use in hydrogenolysis/hydrogenation reactions is also described.

Prior to activation, the hydrogenolysis/hydrogenation catalyst may include about 35 wt % to about 85 wt % CuO, about 8 wt % to about 20 wt % CaO, about 10 wt % to about 30 wt % SiO₂, and about 0.1 wt % to about 5 wt % Na₂O. In other embodiments and prior to activation, the calcined hydrogenolysis/hydrogenation catalyst may include CuO from about 60 wt % to about 70 wt %, CaO from about 10 wt % to about 15 wt %, SiO₂ from about 15 wt % to about 25 wt %, and Na₂O from about 0.5 wt % to about 2 wt %. In yet other embodiments and prior to activation, the calcined hydrogenolysis/hydrogenation catalyst includes about 35 wt % to about 70 wt % CuO, about 8 wt % to about 16 wt % CaO, about 15 wt % to about 22 wt % SiO₂, about 0.1 wt % to about 0.75 wt % Na₂O, about 0.5 to about 1.5 wt % Al₂O₃, and about 0.1 wt % to about 0.75 wt % K₂O. In some embodiments, prior to activation, the calcined hydrogenolysis/hydrogenation catalyst may include Na₂O from about 0.5 wt % to about 1 wt %, or from about 0.5 wt % to less than about 1 wt %.

In the methods, the hydrogenolysis/hydrogenation catalysts are substantially free of chromium. While in some embodiments, the calcium silicate catalysts may include manganese, in other embodiments, the catalyst is substantially free of manganese.

In the methods, the Brunauer-Emmett-Teller Surface Area (BET SA) of the powder of the catalyst may be from about 20 m²/g to about 100 m²/g. In some embodiments, the BET SA is from about 5 m²/g to about 85 m²/g, or from about 10 m²/g to about 85 m²/g, or from about 15 m²/g to about 80 m²/g.

As with the catalyst materials described above, the calcined hydrogenolysis/hydrogenation catalyst from the method, exhibits an XRD pattern indicative of CuO and an additional crystallite phase selected from the group consisting of cubic SiO₂, rhombohedral calcium carbonate CaCO₃, anorthic calcium silicate CaSiO₃, calcium silicate hydroxide hydrate (Ca₁₄Si₂₄O₅₈(OH)₈·2H₂O ), calcium silicate hydrate (4CaO·5SiO₂·5H₂O ), alumina, and combinations of two or more thereof. Further, the calcined hydrogenolysis/hydrogenation catalyst may exhibit a CuO crystallite size of about 50 Å to less than about 240 Å, or exhibits a CuO crystallite size of about 50 Å to about 175 Å. Moreover, the calcined hydrogenolysis/hydrogenation catalyst may exhibit a calcium silicate hydrate crystallite size of about 550 Å to less than about 673 Å, or about 550 Å to about 650 Å.

In another aspect, a method of hydrogenating/hydrogenolysis of a carbonyl-containing organic compound is provided. The method includes contacting the carbonyl-containing organic compound with an activated catalyst that is any of the calcined hydrogenolysis/hydrogenation catalysts described herein. In various embodiments, the carbonyl-containing organic compound may include a ketone, an aldehyde, and/or an ester. In some embodiments, it is a fatty acid ester. More specifically, in any embodiment disclosed herein, the carbonyl-containing organic compound may include, but is not limited to, a fatty acid methyl ester (e.g. C₈-C₂₀ carbon chain), fatty acid wax ester (e.g. C₁₈-C₄₀ carbon chain), furfural, methyl phenyl ketone, di-methyl or di-ethyl esters, or a mixture of any two or more thereof. The hydrogenation/hydrogenolysis may be carried out in a slurry phase reactor that may be a batch reactor, a continuously stirred tank reactor, a tower reactor, or a column reactor.

According to some embodiments, the method may further include reducing the calcined hydrogenolysis/hydrogenation catalyst in a hydrogen atmosphere to obtain a pre-reduced (activated) calcined hydrogenolysis/hydrogenation catalyst. In some embodiments, the reducing may be carried out in the presence of a solvent for a time, and at a temperature, sufficient to reduce the calcined hydrogenolysis/hydrogenation catalyst.

The present invention, thus generally described, will be understood more readily by reference to the following examples, which are provided by way of illustration and are not intended to be limiting of the present invention.

### EXAMPLES

Comparative Examples. A commercial catalyst containing copper and chromium (Cu—Cr) was supplied by BASF (Cu 1950P (a copper chromite catalyst with 36 wt % Cu and 32 wt % Cr)).

A Cu—Al—O catalyst (comparative example) was prepared according to U.S. Pat. No. 6,455,464 B1. A copper nitrate solution (1640 g; 15.48% Cu) was diluted with deionized water to 2500 ml. Sodium aluminate (815.6 g, 25% Al₂O₃) was dissolved and diluted with deionized water to 2500 ml. The copper nitrate and sodium aluminate solutions were then simultaneously to a 12-liter reactor containing deionized water (2500 ml), at a rate of about 33 ml per minute. Sodium carbonate powder (318 g dissolved in 1500 ml deionized water) was added to maintain the reactor at a pH of about 7.4. The catalyst then precipitates from solution at room temperature, and the precipitate is collected by filtration as a filter cake. The cake was then washed with deionized water (3000 ml), three or more times. After drying the washed caked at 120° C. overnight, the CuAl powder was calcined at 700° C.-800° C. for 2 hours.

The reference catalysts were then used to illustrate the superior performance of the inventive catalyst when applied to fatty ester hydrogenolysis.

Example 1. Preparation of CuOCaSiO₃ Catalyst. Cu(NO₃)₂ solution (1756 g, 16.2 wt % Cu) was diluted with deionized water to a total volume of 1800 ml in a reactor with a mixing paddle, and the mixing speed was set at 800 RPM (round per minute). Calcium silicate (237.5 g; LOI=16%; “MicroCel E®” (a synthetic calcium silicate hydrate available from Imerys)) was slowly added to the Cu(NO₃)₂ solution. Agitation was continued until the calcium silicate was entirely dispersed. Sodium carbonate (700 g; “soda ash”) was dissolved in deionized water (3 liters) to form a solution that was then added to the Cu(NO₃)₂ and calcium silicate slurry to maintain a pH of about 7, at room temperature. Over about 1 hour a precipitate forms and is collected by filtration as a filter cake. The cake is then washed with deionized water, followed by drying and calcination at 500° C. for 2 hours to form the catalyst. The catalyst has about 60 wt % CuO and the balance is CaSiO₃ and others traces. Chemical analysis: CuO 63.1 wt %, CaO 13.9 wt %, SiO₂ 21.8 wt %, and Na₂O 1.4 wt %. Loose ABD (apparent bulk density): 0.2 g/ml; Packed apparent bulk density (ABD): 0.3 g/ml. Particle size distribution (PSD) is shown in Table 1 under sonication or no sonication.

TABLE 1
Particle Size Distribution of Example 1.

| Sonication Time | D₁₀ (μm) | D₅₀ (μm) | D₉₀ (μm) |
|---|---|---|---|
| No sonication | 8.0 | 35.0 | 404.4 |
| Sonicated for 1 min | 4.7 | 22.3 | 43.9 |
| Sonicated for 3 min | 4.2 | 20.8 | 40.8 |

Example 2. Pilot plant procedure for CuOCaSiO₃ Catalyst. Cu(NO₃)₂ solution (31.6 kg; 16.2 wt % Cu) was diluted to 25 L with deionized water in a reactor with a mixer set at about 800 RPM. Calcium silicate (4.275 kg; LOI=16%; “MicroCel E®”) was slowly added to the Cu(NO₃)₂ solution. Agitation was continued until the calcium silicate was entirely dispersed. Sodium carbonate (10.8 kg; “soda ash”) was dissolved in deionized water (40 liters) with NaOH (3.6 kg) to form a solution. The soda ash and NaOH solution was then added to the Cu(NO₃)₂ and calcium silicate slurry to maintain a pH of about 7, at room temperature. Over about 1 hour a precipitate forms and is collected by filtration as a filter cake. The cake is then washed with deionized water, followed by drying (120° C.). The PSD of the material is D₁₀ 9.7 μm, D₅₀ 26.6 μm, and D₉₀ 66.4 μm.

Examples 3-8. The powders from Example 2 was subjected to different calcination temperatures according to Table 2. In each Example, powder from Example 2 was calcined in a muffle furnace by heating to the temperature indicated over 1 hour and then held at temperature for 2 hours prior to cooling to room temperature.

TABLE 2
Examples and calcination temperatures.

| Example | Calcination Temperature (° C.) |
|---|---|
| 3 | 500 |
| 4 | 600 |
| 5 | 650 |
| 6 | 700 |
| 7 | 750 |
| 8 | 800 |

Examples 9-12. The catalyst from Example 1 was prepared with different sodium content. These catalysts have about the same composition: 63.1 wt % CuO, 13.9 wt % CaO and 21.8 wt % SiO₂, after calcination at about 500° C. for 12 hours. The only difference is Na₂O content in the catalyst due to differences in washing with varying amount of deionized water. The results are shown in Table 3.

TABLE 3
Sodium Content of various Examples.

| | Wash volume (deionized | Na Content (as |
|---|---|---|
| Example | water; liters) | Na₂O; wt %) |
| 9 | 15 | 1.4 |
| 10 | 18 | 1.0 |
| 11 | 21 | 0.7 |
| 12 | 24 | 0.59 |

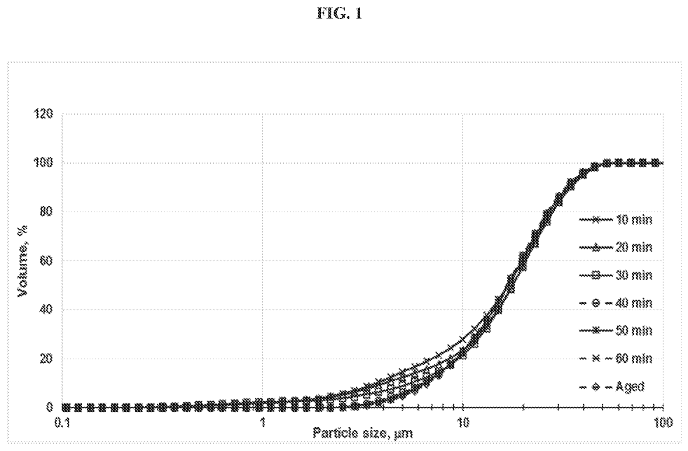

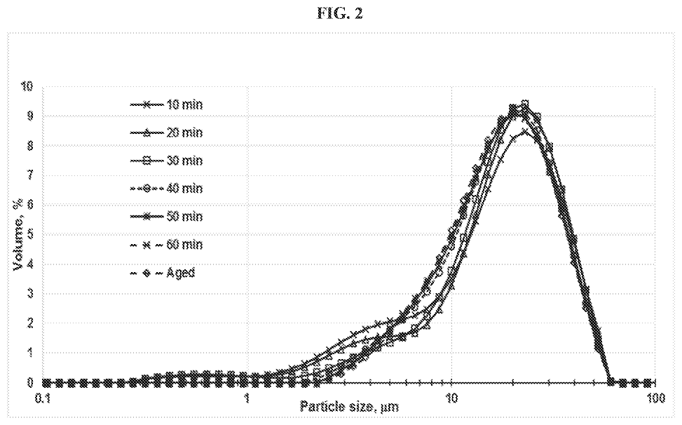

FIGS. 1 and 2 illustrate how the catalyst particle size is formed and changed during the precipitation of Example 1. Each plot in FIG. 1 shows the Cumulative Volume % (vol %) distribution of particles detected via laser diffraction within a particle size range. Each plot in FIG. 2 shows a normal distribution curve in which a majority of the sample volume is occupied by the mid-range sized particles (i.e., Volume %, vol %). According to the plots, the catalyst should have a PSD of D₁₀ 7.7 μm, D₅₀ 19.1 μm, and D₉₀ 37.5 μm. However, it is clear that the average particle size distribution depends on precipitation conditions.

BET Surface Area Measurement. BET (Brunauer-Emmett-Teller) surface area measurement was performed by following ASTM method D3663-03 Standard Test Method for Surface Area of Catalysts and Catalyst Carriers. Some of the catalyst BET surface area are summarized in the Table 4.

TABLE 4
BET Surface Areas for various Examples.

| | Calcination | BET Surface |
|---|---|---|
| Example | Temperature (° C.) | Area (m²/g |
| 3 | 500 | 81.2 |
| 4 | 600 | 67.8 |
| 5 | 650 | 58.6 |
| 6 | 700 | 33.9 |
| 7 | 750 | 14.3 |
| 8 | 800 | 3.4 |

It is clear from Table 4 that higher calcination temperatures result in lower BET surface area. The copper silicate catalysts having a BET SA from 14.3 to 82 m²/g all exhibit good fatty alcohol yields from the hydrogenolysis methyl esters. Further calcination at higher temperature, such as 800° C., results in very low BET SA, 3.4 m²/g and a lower activity.

XRD Analysis of Selected Catalysts from Examples 3-8. XRD analysis of Examples 3-8 (calcined from 500° C. to 800° C.) were performed to identify the crystallite phases and crystallite sizes. XRD analysis were performed according to the procedure described here. An Empyrean diffraction system with a copper anode tube was operated with generator settings at 45 kV and 40 mA to produce Cu K_{α1} radiation of wavelength 1.54060 Å used to generate XRD analytical data. The optical path consisted of a 0.04 rad primary soller slit, 15 mm beam mask, 1° divergence slit, 2° anti-scatter slit, the sample, a monochromator, a secondary 0.02 rad soller slit and an X'Celerator position sensitive detector.

The sample catalyst was ground to a fine powder using a mortar and pestle and then backpacked into a round mount sample holder. The sample holder is loaded onto a sample spinner during data acquisition to improve particle counting statistics. The data collection from the round mount covered a range from 15° to 90° 2θ using a continuous scan with a step size of 0.017° 2θ and a time per step of 400 seconds. A graphite monochromator was used to strip unwanted radiation, including Cu K_{β} radiation. Panalytical HighScore version 4.5 software and ICDD PDF 4+ 2020 version powder diffraction file database was used for phase identification analysis. Highscore was also used for profile fitting to determine d-spacing, FWHM and peak positions used to calculate crystallite size estimates using the Scherrer equation. The details of XRD patterns and crystallite size of sample from each example are shown below.

Example 3 calcined at 500° C. exhibited major peaks that are fit well as monoclinic copper oxide, (CuO). Several smaller remaining peaks are fit well as rhombohedral calcium carbonate (CaCO₃). Candidates for the few remaining minor peaks are calcium silicate hydrate (4CaO·5SiO₂·5H₂O ), cubic silica (SiO₂), and/or a phase of alumina (Al₂O₃). Copper oxide crystallite size was estimated based on the (111) reflection at about 58 Å.

Example 5 calcined at 650° C. exhibited major peaks that are fit well as monoclinic copper oxide (CuO). Candidates for the few remaining minor peaks are rhombohedral calcium carbonate (CaCO₃), calcium silicate hydrate (4CaO·5SiO₂·5H₂O ), calcium silicate hydrate (Ca₂SiO₄·H₂O), and/or cubic silica (SiO₂). Copper oxide crystallite size was estimated based on the (111) reflection at about 80 Å.

Example 7 calcined at 750° C. exhibited major peaks that are fit well as monoclinic copper oxide (CuO) and anorthic calcium silicate (CaSiO₃). Calcium silicate hydrogen oxide (Ca₂SiO₄·0·3H₂O) may fit some very small trace peaks. The copper oxide crystallite size was estimated based on the (111) reflection at about 149 Å. The calcium silicate crystallite size was estimated based on the (220) reflection at 573 Å.

Example 8 calcined at 800° C. exhibited major peaks that are fit well as monoclinic copper oxide (CuO) and anorthic calcium silicate (CaSiO₃). Calcium silicate hydrogen oxide (Ca₂SiO₄·0·3H₂O) may fit some very small trace peaks. The copper oxide crystallite size was estimated based on the (111) reflection at about 240 Å. The calcium silicate crystallite size was estimated based on the (220) reflection at 673Å.

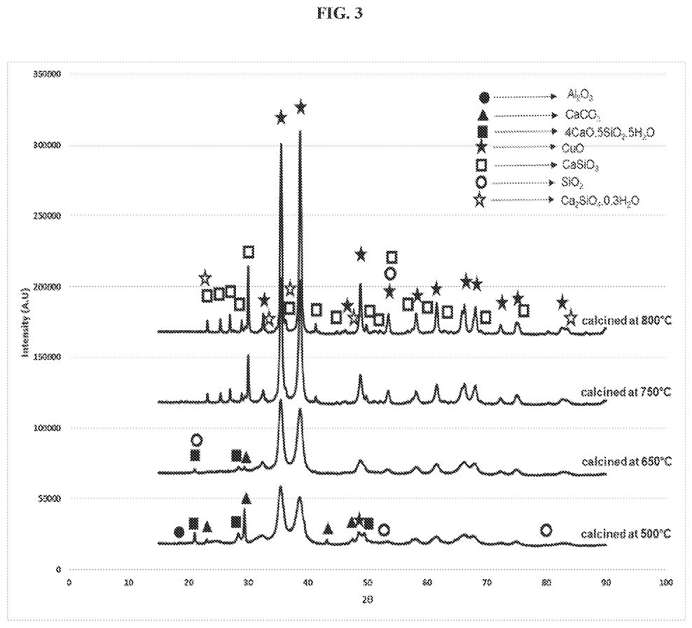

The XRD analyses of these catalysts show that the catalysts contain CuO and one or more of the following crystallite phases: cubic SiO₂, rhombohedral calcium carbonate (CaCO₃), anorthic calcium silicate (CaSiO₃), Truscottite (calcium silicate hydroxide hydrate; Ca₁₄Si₂₄O₅₈(OH)₈·2H₂O ), Torbermorite (calcium silicate hydrate; 4CaO·5SiO₂·5H₂O ), and alumina. These are shown in FIG. 3.

It is also shown that the crystallite phases transform as the calcination temperature changes. With increasing calcination temperature, CuO crystallite size increases from about 58 to about 240 Å. Table 5 shows the effects of calcination temperatures on CuO crystallite sizes. The CuO crystallite size was estimated based on the (111) reflection. Calcium silicate hydrate also starts to dehydrate and eventually forms anorthic calcium silicate, CaSiO₃, with increasing temperature. The crystallite size of calcium silicate increases from 573 to 673 Å, as the calcination temperature changes from 750° C. to 800° C.

TABLE 5
Effect of Calcination
Temperature on CuO Crystallite Size.

| | Calcination | CuO Crystallite |
|---|---|---|
| Example | Temperature (° C.) | Size, Å |
| 2 | 500 | 58 |
| 5 | 650 | 80 |
| 7 | 750 | 149 |
| 8 | 800 | 240 |

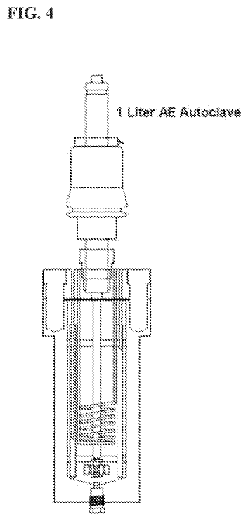

Catalyst Performance. The testing procedure. Catalytic activity and selectivity of the catalysts were evaluated by slurry phase hydrogenolysis of a methyl ester to a fatty alcohol. Catalyst performance evaluations were performed for both methyl ester hydrogenolysis and wax ester hydrogenolysis in a one-liter autoclave (Illustrated in FIG. 4).

TABLE 6
Reaction Conditions for Testing of Catalyst Performance.

| | |
|---|---|
| Temperature (° C.) | 280 |
| Total Pressure, psig | 2500 |
| Agitation Rate, rpm | 2000 |
| Catalyst Loading, wt % | 0.8 |

TABLE 7
Feedstock compositions for Testing of Catalyst Performance.

| Material | Amount (wt %) |
|---|---|
| C-8 ME | 0.00 |
| C-10 ME | 0.00 |
| C-12 ME | 74.8 |
| C-14 ME | 23.1 |
| C-16 ME | 0.6 |
| C-18:0 ME | 0.00 |
| C-18:1 ME | 0.00 |
| C-18:2 ME | 0.00 |
| C24 WE | 0.6 |
| 12:14 + 14:12 WE | 0.6 |

Note:
ME: methyl ester;
WE: wax ester

Procedure for methyl ester hydrogenolysis: The catalyst (0.8 wt %) is loaded into the reactor through the opening the top screw of the reactor head. 452 g of C12-C14 fatty acid methyl ester is loaded through the funnel located on the gas line which is used for pressurization and hydrogen gas feed. The autoclave system was purged with N₂ few times to remove air and then purged with hydrogen few times. Agitation was set to 2000 rpm and the temperature of the furnace was ramped to temperature, and the autoclave was jacketed (a typical ramping rate was 3° C/min). At 280° C., the autoclave was pressurized with H₂ to 2500 psi, and designated as start time “0”. Every hour for 5 hours a 5 ml liquid sample was collected through the port with the frit at the tip inside the autoclave. The sample was then analyzed by GC. Total fatty alcohol yield was calculated summing the fatty alcohol concentrations from the GC analysis.

Procedure for wax ester hydrogenolysis:

TABLE 8
Reaction Conditions for Fatty Acid Wax Ester Hydrogenolysis.

| | |
|---|---|
| Temperature (° C.) | 300 |
| Total Pressure, psig | 4350 |
| Agitation Rate, rpm | 1500 |
| Catalyst Loading, wt % | 0.75 |
| Feed | C16-C18 fatty acid (27 |
| | wt % C16 fatty acid; 72 |
| | wt % C18 fatty acid) |
| Heel | 454 g C12—C14 Fatty |
| | alcohol |
| Run | 6 hours, each hour injection |
| | of 55 g of fatty acid for |
| | hours 0, 1, 2, and 3. |

The catalyst is loaded through opening the top screw of the reactor head. 454 g of Cu₁₂-C₁₄ fatty alcohol is loaded through the funnel located on the gas line which is used for pressurization and hydrogen gas feed. The autoclave was purged with N₂ few times to remove air and then purged with hydrogen few times. The agitation at 1500 rpm was started and the furnace ramped (typical ramping rate 3° C/min) to temperature in a jacketed autoclave. At 300° C., the autoclave was pressurized with hydrogen to 4350 psi, 55 g of C₁₆-C₁₈ fatty acid was injected through the pump. This was designated as start time “0”. 5 ml of liquid sample was collected through the port with the frit at the tip inside the autoclave at 1st hr. Then another 55 g of C₁₆-C₁₈ fatty acid was injected. At the 2^{nd} and 3^{rd} hour, this sampling and injection of fatty acid was be continued. The experiment was continued to 6 hours without further injection of fatty acid after 3 HOS (hour-on-steam). Each sample (total 6 samples) was analyzed be gas chromatography (“GC”).

The SAP (saponification) value of each sample was calculated by wet titration, and fatty acid conversion was calculated based on the % reduction in SAP: Conversion (%)=(SAP value in resulting feed mixture-SAP value in the product)x*100/SAP value in resulting feed mixture. For each experiment, the final hour sample was also analyzed by GC FID to measure the concentrations of fatty alcohols and by-products.

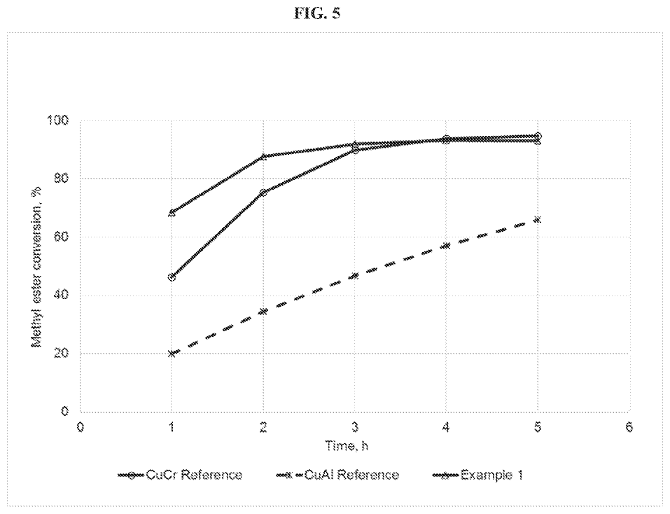

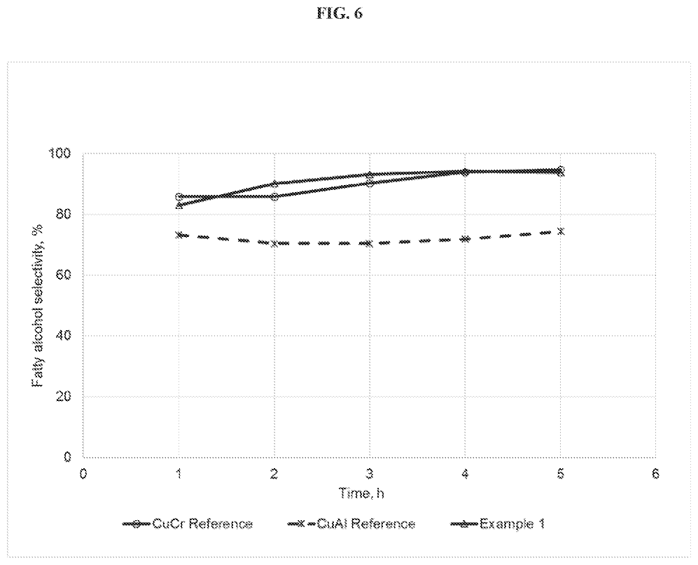

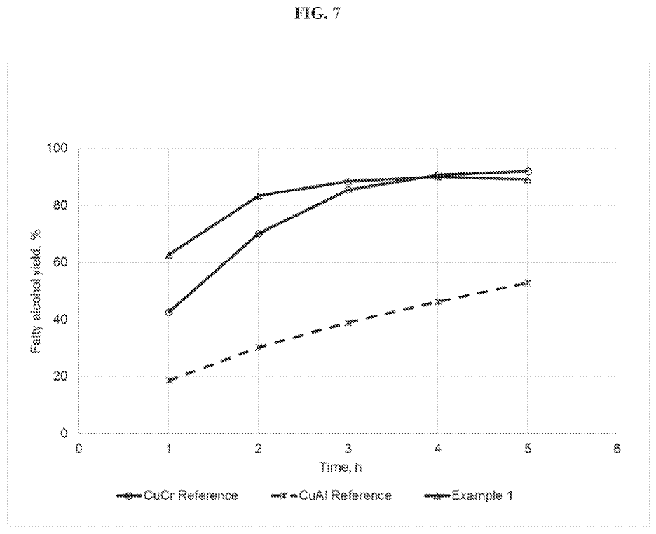

Comparison of catalyst performance of commercial CuCr and CuAl versus the CuO—CaSiO₃ catalysts. Under the described testing conditions, for methyl ester hydrogenolysis, CuAl powder catalyst underperforms the commercially used CuCr powder catalyst. However, the CuO—CaSiO₃ catalysts, described herein, has significantly better performance. This is illustrated in FIGS. 5-7. As the reaction time progresses, the desired product fatty alcohol for the CuO—CaSiO₃ is always higher than CuCr reference catalyst throughout the test time period. These results demonstrate that CuO—CaSiO₃ powder catalyst is suitable to substitute CuCr powder catalyst for fatty methyl ester hydrogenolysis application.

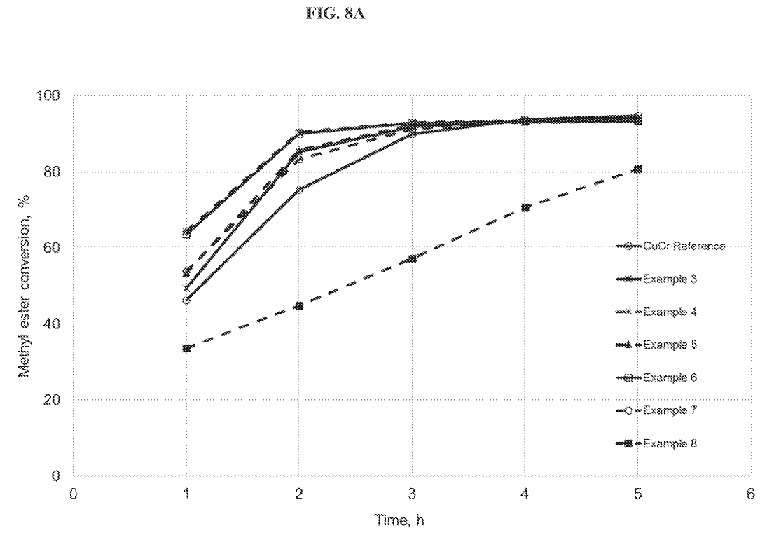

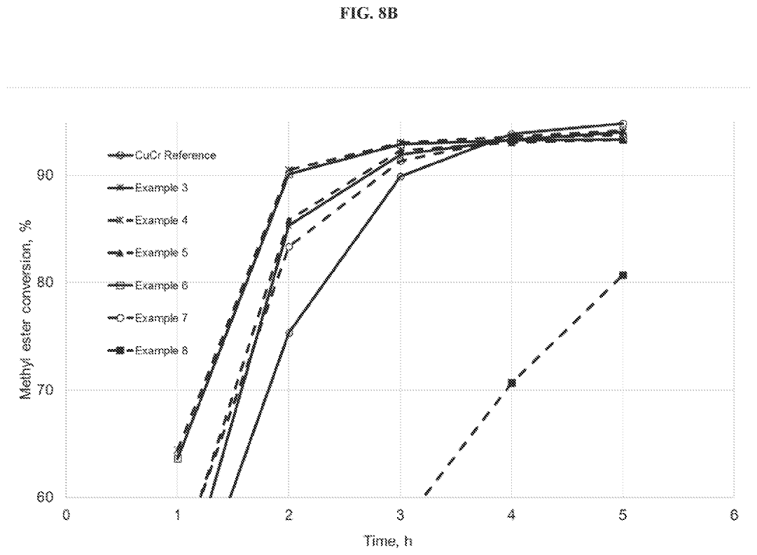

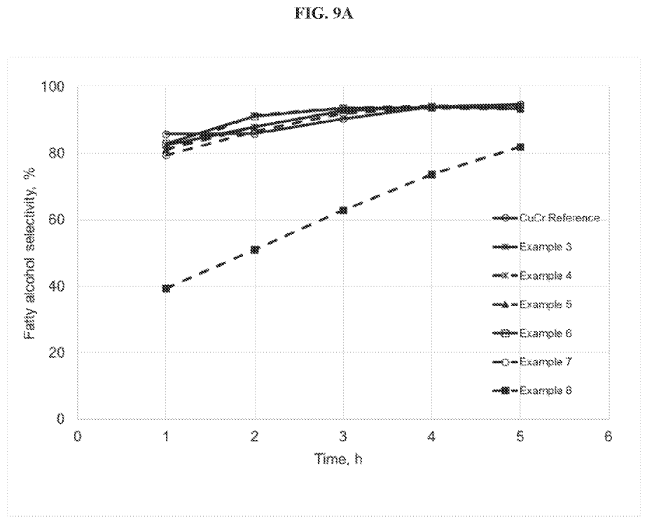

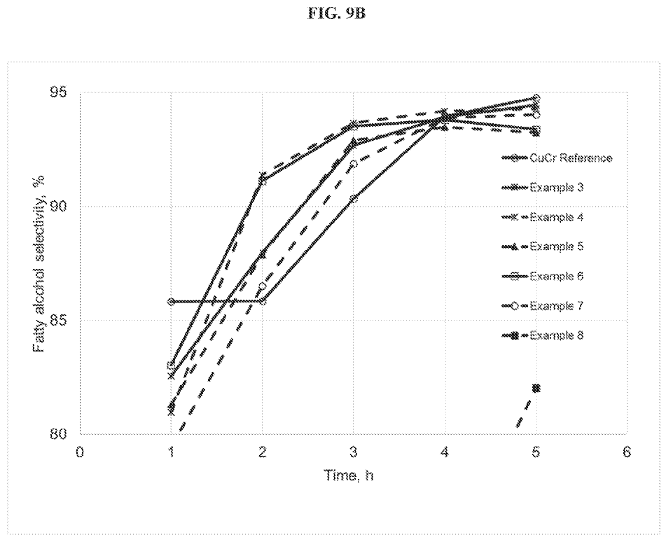

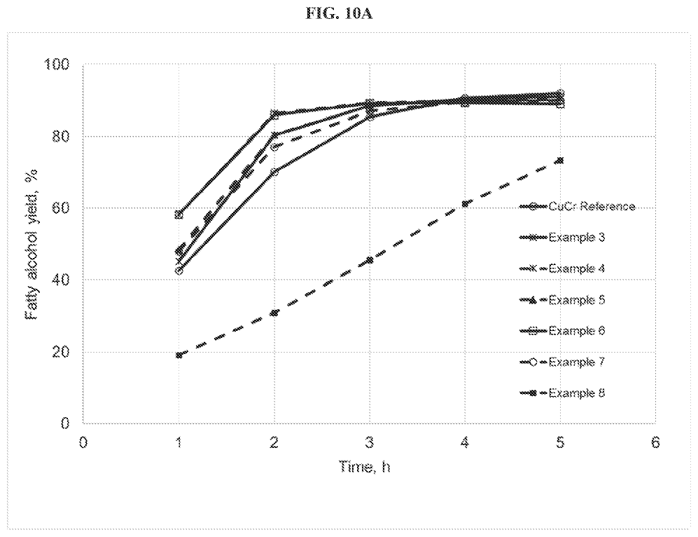

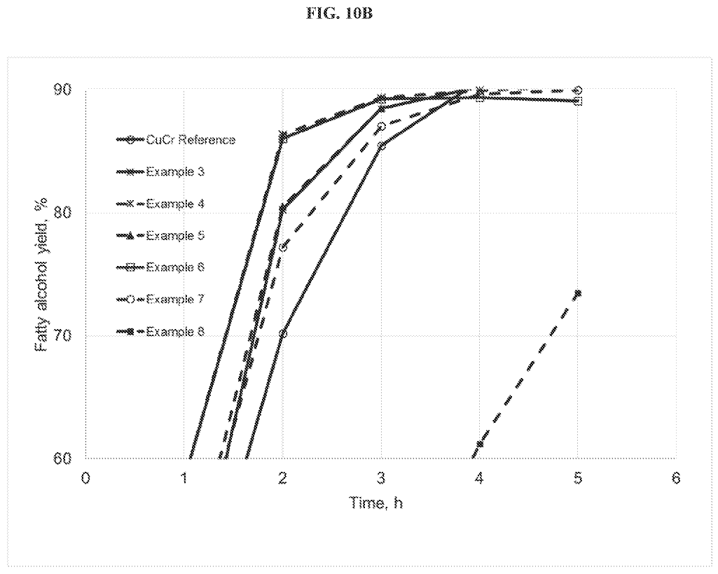

Impact of BET Surface Area on Catalytic Performance. Low calcination temperatures result in higher BET surface area and therefore higher hydrogenation/hydrogenolysis activity. However, a high BET SA powder catalyst is more susceptible to chemical attack, especially from acids in the feed stock, leading to greater metal leaching into the hydrogenolysis product. When the catalyst is calcined at a high enough temperature, the catalyst BET surface area will decrease and a more stable catalyst is formed that is stable under the reaction conditions, i.e. for feed stocks that contain some acid. The catalyst powders were calcined at different temperatures in Examples from 3 to 8 that produced the powders with different BET SA. These samples were tested for methyl ester hydrogenolysis catalytic performance. The results are shown in the FIGS. 8-10. The product alcohol yield curves show that catalysts made by calcination temperature ranging from 500° C. to 750° C. all have good alcohol yield. These results show that catalysts with broad range of BET surface area, from 14 to 80 m²/g, provide for good catalyst performance. However, calcination at 800° C. resulted in significantly lower BET surface area (3.4 m²/g) and larger CuO crystallite size (240 Å), thereby producing poor catalyst performance with low alcohol yield. From the above, it is clear that the CuO—CaSiO₃ catalysts have higher activity than the current state of the art CuCr catalysts.

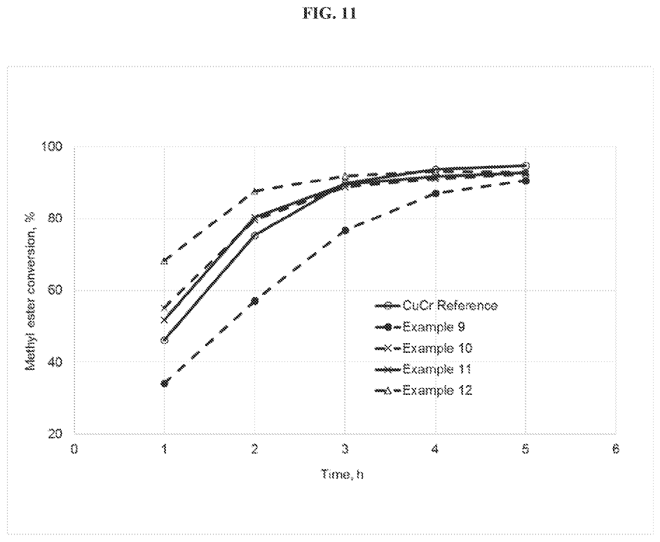

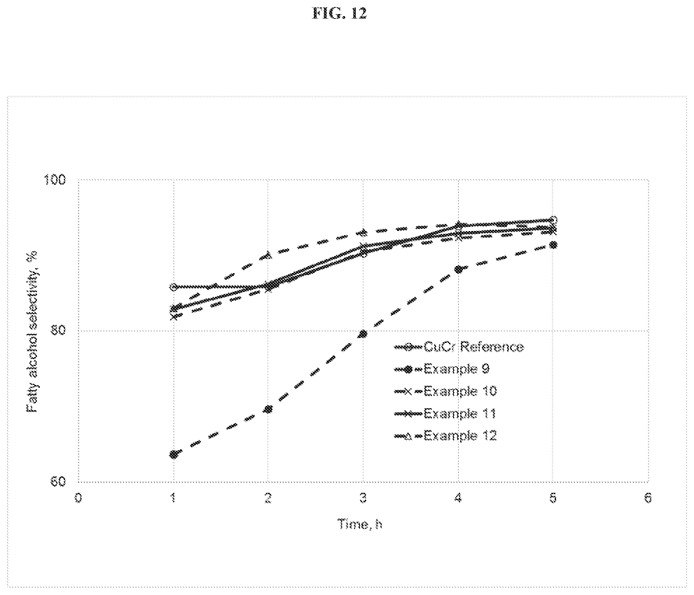

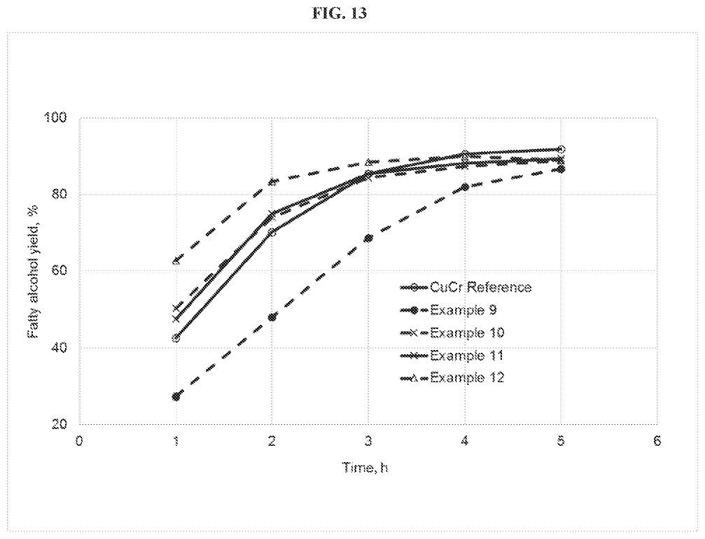

Effects of Sodium Oxide Contents on Catalyst Performance. A series of catalysts with different sodium oxide contents from Examples 9-12 were evaluated for the slurry phase methyl ester hydrogenolysis application. As shown in FIGS. 11-13, the catalyst from Example 9 catalyst has Na₂O at 1.4 wt %, and the catalyst exhibits lower activity and selectivity than commercially used CuCr reference. With further washing to remove residual sodium the catalyst performance increases. Example 12 catalyst has the highest activity with the highest rate of making fatty alcohol product throughout the testing period.

Wax Ester Hydrogenolysis Performance Comparison. For catalyst performance comparison, the liquid product during the reaction was collected for SAP (saponification) value analysis. SAP value is the hydrolysis of ester with KOH (or NaOH) to form alcohol and potassium or sodium salt of the corresponding acid. Higher SAP value means a higher ester content. In this case higher SAP value means that a lower amount of the ester is converted to alcohol, i.e. the catalyst activity is lower. The performance of the CuO—CaSiO₃ catalysts for wax ester hydrogenolysis performance were compared to the state of the art CuCr catalysts. In this series of tests, the hydrogenolysis product was withdrawn every hour for analysis, following by injection of new fatty acid as a feed in the first four hours.

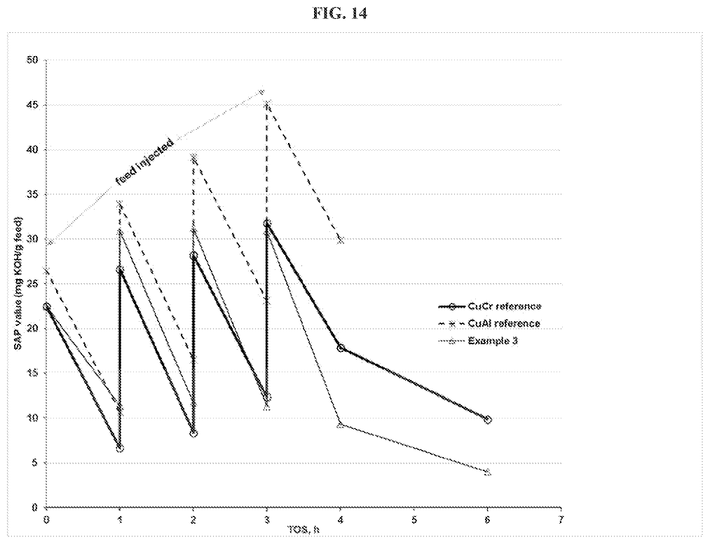

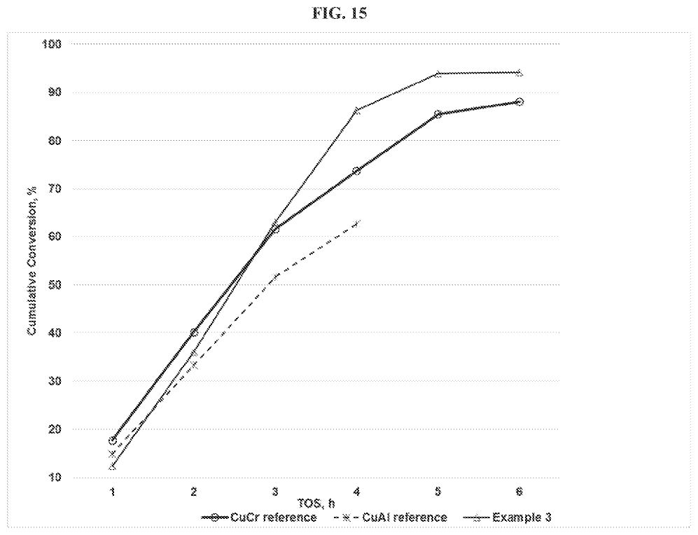

The results are summarized in FIGS. 14 and 15. The figures show that after 4^{th} injection, and as the reaction time continues, the residual wax ester diminishes, and so does the ester conversion as the system approaches equilibrium. The Example 3 catalyst (calcined at 500° C.) has consistently higher wax ester conversion than the CuCr reference catalyst starting with the 2^{nd} hour of testing (FIG. 14 and Table 9). The higher conversion during the first hour for the CuCr catalyst could be attributed to a more facile reduction process with that catalyst. Overall, the CuO—CaSiO₃ catalysts exhibit significantly higher activity and alcohol yield.

TABLE 9
Comparison of CuO—CaSiO₃
Catalyst Activity versus a CuCr Catalyst

| Total Cumulated | Final SAP | Overall |
|---|---|---|
| Feed SAP 67.9 | value at 6 h | Conversion, % |
| CuCr reference | 8.1 | 88.1% |
| Example 3 | 4.0 | 94.1% |

Another advantage of the CuO—CaSiO₃ Catalyst is its better selectivity to fatty alcohols, and making less hydrocarbon by-product impurities in slurry phase wax ester process. As shown in Table 10 the new CuO—CaSiO₃ catalysts produces less dodecane, tetradecane, hexadecane, and octadecane compared to the CuCr catalysts. All of these alkanes are the over-hydrogenated by-product resulting in low product purity and yield.

TABLE 10
Hydrocarbon Impurities formation in Final Products
after 6 hours of Reaction Time

| | | Tetra- | Hexa- | Octa- |
|---|---|---|---|---|
| | Dodecane, | decane, | decane | decane |
| Catalyst | % | % | % | % |
| CuCr Reference | 0.27 | 0.05 | 0.02 | 0.03 |
| Example 3 | 0.14 | 0.03 | 0.01 | 0.03 |

Catalyst Filtration Properties. Catalyst separation experiments (both centrifuge separation and filtration) of the spent slurry in slurry phase methyl ester process were conducted to compare the catalyst filterability. The results show that the CuO—CaSiO₃ catalysts have comparable separation properties to state of the art catalysts.

It is a qualitative estimate on settling and separation by centrifugation at 11000 rpm for 5 minutes and take picture for visual comparison. At first, the spent catalyst slurry was stirred with the liquid products and unreacted fatty acid methyl ester (0.8 wt % catalyst loading) for 5 mins. Then, the top 35 mL liquid was collected and a picture obtained to see if any particles were floating. The separation efficiency is measured qualitatively based on the color and fine particles floating in the collected liquid.

Centrifuge separation tests show that both a CuO—CaSiO₃ catalyst and a CuCr provide clear liquid products. To further confirm this, the liquid products were analyzed for metal leaching from catalysts. As shown below, both CuO—CaSiO₃ catalysts and CuCr catalysts have low metal leaching (Table 11). This indicates that the CuO—CaSiO₃ catalysts have good chemical stability under the reaction condition.

TABLE 11
Metal Contents in Reaction
Liquid Product after Separation

| | HOS | Ca, | Cr, | Cu, | Mn, | Si, |
|---|---|---|---|---|---|---|
| | sample | ppm | ppm | ppm | ppm | ppm |
| CuCr | 6 | | <1 | <1 | <1 | |
| reference | | | | | | |
| Example 3 | 6 | <1 | | <1 | | <1 |

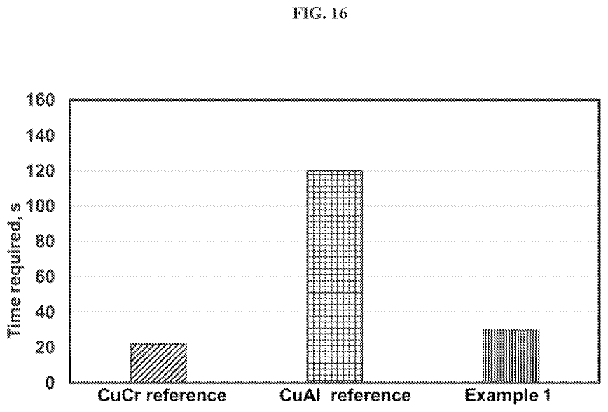

Procedure for Filtration. Spent catalyst slurry (12 ml; well mixed) was loaded into a syringe. A syringe filter (0.45 μm) was affixed in the tip of the syringe, a timer was started when the plunger of the syringe was first actuated. The filtration was stopped and the filter changed if there was no filtrate coming out with a maximum hand pressure. After filter 12 mL of the slurry, the timer was stopped. This test qualitatively estimates the ease of filtration (by time required for making same amount of filtrate) using a 0.45 μm filter). Some results are shown in FIG. 16, and show that the CuO—CaSiO₃ catalysts have a similar filtration time to the state of the art catalysts.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A hydrogenolysis/hydrogenation catalyst comprising:
copper oxide;
calcium oxide;
silicon dioxide; and
sodium oxide;
wherein:
the hydrogenolysis/hydrogenation catalyst is a powder;
the hydrogenolysis/hydrogenation catalyst is substantially free of chromium; and
the hydrogenolysis/hydrogenation catalyst exhibits a crystallite phase of CuO and an additional crystallite phase selected from the group consisting of cubic SiO₂, rhombohedral calcium carbonate CaCO₃, anorthic calcium silicate CaSiO₃, calcium silicate hydroxide hydrate (Ca₁₄Si₂₄O₅₈(OH)₈·2H₂O ), calcium silicate hydrate 4CaO·5SiO₂·5H₂O , alumina, and combinations of two or more thereof.

2. The hydrogenolysis/hydrogenation catalyst of claim 1, wherein the powder has a d₁₀% particle size of about 1 μm to about 10 μm.

3. The hydrogenolysis/hydrogenation catalyst of claim 1, wherein the powder has a d₅₀% particle size of about 10 μm to about 25 μm.

4. The hydrogenolysis/hydrogenation catalyst of claim 1, wherein the powder has a d₉₀% particle size of about 30 μm to about 45 μm.

5. The hydrogenolysis/hydrogenation catalyst of any one of claim 1, wherein the hydrogenolysis/hydrogenation catalyst comprises CuO from about 35 wt % to about 85 wt %, CaO from about 8 wt % to about 20 wt %, SiO₂ from about 10 wt % to about 30 wt %, and Na₂O from about 0.1 wt % to about 5 wt %.

6. The hydrogenolysis/hydrogenation catalyst of claim 1, wherein prior to activation, the hydrogenolysis/hydrogenation catalyst comprises Na₂O from about 0.5 wt % to about 1 wt %.

7. The hydrogenolysis/hydrogenation catalyst of claim 1, wherein prior to activation, the hydrogenolysis/hydrogenation catalyst is substantially free of manganese.

8. The hydrogenolysis/hydrogenation catalyst of claim 1, wherein the hydrogenolysis/hydrogenation catalyst exhibits a Brunauer-Emmett-Teller (“BET”) surface area of about 5 m²/g to about 85 m²/g.

9. The hydrogenolysis/hydrogenation catalyst of claim 1, wherein the hydrogenolysis/hydrogenation catalyst exhibits CuO and an additional crystallite phase selected from the group consisting of cubic SiO₂, rhombohedral calcium carbonate CaCO₃, anorthic calcium silicate CaSiO₃, calcium silicate hydroxide hydrate (Ca₁₄Si₂₄O₅₈(OH)₈·2H₂O ), calcium silicate hydrate 4CaO·5SiO₂·5H₂O, and combinations of two or more thereof.

10. The hydrogenolysis/hydrogenation catalyst of claim 1, wherein the hydrogenolysis/hydrogenation catalyst exhibits a CuO crystallite size of about 50 Å to less than about 240 Å.

11. A method of preparing a hydrogenolysis/hydrogenation catalyst, the method comprising:
mixing a copper-containing material and silicate-containing material in a solution;
adding a caustic material to form an aqueous slurry comprising a precipitate;
collecting the precipitate;
drying the precipitate to form a dried precipitate; and
calcining the dried precipitate to form a calcined hydrogenolysis/hydrogenation catalyst;
wherein:
the calcined hydrogenolysis/hydrogenation catalyst is a powder; and
the calcined hydrogenolysis/hydrogenation catalyst is substantially free of chromium.

12. The method of claim 11, wherein the aqueous slurry comprises a pH of about 6.0 to about 9.0.

13. The method of claim 11, wherein the collecting comprises filtering the aqueous slurry to remove the precipitate.

14. The method of claim 11, wherein the calcining is conducted at a temperature of about 400° C. to below about 800° C.

15. (canceled)

16. The method of claim 11, wherein the calcining is conducted for about 10 minutes to about 10 hours.

17. (canceled)

18. The method of claim 11, wherein the copper-containing material is a copper salt comprising copper nitrate, copper sulfate, copper chloride, copper bromide, copper acetate, or a combination of any two or more thereof

19. The method of claim 11, wherein the silicate-containing material comprises a silicate salt comprising calcium silicate.

20. The method of claim 11, wherein the caustic material is Na₂CO₃, NaOH, K₂CO₃, KOH, or a combination of any two or more thereof.

21.-40. (canceled)

41. A calcined hydrogenolysis/hydrogenation catalyst prepared according to the method of claim 11.

42. A method of hydrogenating a carbonyl-containing organic compound, the method comprising contacting the carbonyl-containing organic compound with the hydrogenolysis/hydrogenation catalyst of claim 31.

43.-47. (canceled)
